(19) United States
(12) Patent Application Publication
(10) Pub. No.: US 2012/0114776 A1
(43) Pub. Date: May 10, 2012

(54) METHODS FOR PREPARING PROBIOTIC NANOPARTICLES

(76) Inventor: Janos Feher, Budapest (HU)

(21) Appl. No.: 12/939,395

(22) Filed: Nov. 4, 2010

(51) Int. Cl. A61K 35/74; C12P 19/00; C12P 7/64; C12P 19/34; A61K 35/56; A61P 43/00; A61K 31/70; A61K 38/00; A61K 31/20; C12P 19/14; A61K 35/66; C12P 21/00; A61K 31/7088

(52) U.S. Cl. 424/780; 435/68.1; 435/72; 435/134; 435/91.1; 514/44 R; 514/23; 514/1.1; 514/558

(57) ABSTRACT

Various embodiments of the present invention are directed toward a method for preparing probiotic nanoparticles from natural sources, comprising performing a biological preparation phase such as isolating any cells derived from either prokaryote or eukaryote cells, performing a chemical preparation phase such as performing an enzymatic procedure (or heating, or chemicals) for killing or obtaining cell derived ingredients, performing a physical preparation phase such as performing ultrasonication, and performing a formulation preparation phase such as powderized drying.

### FIELD OF THE INVENTION

The present invention relates to methods for preparing nanoparticles from natural sources and, more particularly, some embodiments relate to methods for preparing nanoparticles from probiotics, and to the use thereof either alone or in combination with pharmacologically compatible compounds for medical, nutritional and cosmetic purposes.

### DESCRIPTION OF THE RELATED ART

Evolutionarily, the human body lives in symbiosis with a complex ecosystem that is composed of more than 10¹⁴ individual bacteria comprising over 500 different species inhabiting the mucous membranes, mainly the intestine, but also the airways and urogenital mucosa, as well as the conjunctiva and the skin. For comparison, the total number of microbes in the human gut exceeds 10 to 100 times the sum of all our cells. This collection of bacteria, known as microflora or most recently as microbiota, is acquired soon after birth and persists throughout life. (Srikanth C V, McCormick B A. Interactions of the intestinal epithelium with the pathogen and the indigenous microbiota: a three-way crosstalk. Interdiscip Perspect Infect Dis. 2008; 2008:626827. Epub 2008 Oct. 29). These microbes have been considered an “extended genome” of millions of microbial genes, and denominated as micro biome. Accumulating evidences have suggested a highly complex cross talk between microbiota and the host immune system. On one hand, host derived mucous substances, including enzymes, like lysozyme; regulate the adherence and survival of microbes on the mucous membranes. On the other hand, certain substances from killed bacteria, including nucleotides, are captured by the host immune system. Recently, extensive studies have been launched to reveal the role of microbiota in both inflammatory diseases of mucous membrane and their contribution to systemic diseases such as the metabolic syndrome (atherosclerosis, type 2 diabetes, obesity, arterial hypertension), neuropsychiatric diseases (anxiety, depression, panic disease), neurodegenerative diseases (Alzheimer's, Parkinson's, age-related macular degeneration) and cancer. (Turnbaugh P J, Gordon J I. The core gut micro biome, energy balance and obesity. Physiol. 2009 Sep. 1; 587(Pt 17):4153-8. Epub 2009 Jun. 2).

Probiotics, a subgroup of the microbiota, are traditionally defined as live microorganisms, which confer a beneficial health effect on the host. Currently, the best-studied probiotics are Lactobacilli, Bifidobacterium and Saccharomyces, although some other organisms used as probiotics in humans include Escherichia coli, Streptococcus, Enterococcus, Bacteroides, and Propionibacterium. One of the main biological functions of probiotics is preventing pathogens' invasion of the host. In normal conditions, host mucous membranes in the gastro-enteral and urogenital tract as well as in airways and conjunctiva contain enzymes (e.g., lysozyme) to kill probiotics and both epithelial cells and macrophages engulf nanoscale fragments of probiotics. This mechanism is essential for the continuous stimulation of the host immune system, which through feedback mechanisms regulates mucous membrane functions. This cross talk between probiotics and immune system is fundamental for maintaining the host-probiotic symbiosis at the mucosal membranes and the adequate immune function systemically. (Resta S C. Effects of probiotics and commensals on intestinal epithelial physiology: implications for nutrient handling. J Physiol. 2009 Sep. 1; 587(Pt 17):4169-74. Epub 2009 Jul. 13).

However, several life-style factors may compromise this symbiosis. Among these the most common are: antibiotic use, medicines, drugs, processed foods, alcohol, smoking and other environmental pollutions. Consequently, the symbiosis between host and probiotics becomes deteriorated which compromises the function of the probiotics to stimulate the host's immune system. This condition, called dysbiosis, may have two consequences: (1) local inflammatory disease of the mucous membranes, for example gastritis, colitis, periodontitis, vaginitis, bronchitis, esophagitis and conjunctivitis; and (2) systemic diseases due to impaired systemic immune functions. Accumulating experimental and clinical data suggest that chronic inflammatory diseases of the mucous membranes, in addition to the local disease, represent core pathogenic contributors to the development of infective diseases, autoimmune diseases, neuropsychiatric diseases, age-related diseases, among them, cardiovascular diseases, type 2 diabetes, Alzheimer's disease, Parkinson's disease, osteoporosis, osteoarthritis, cancer, etc. It should be emphasized that at present time only expensive and symptomatic treatments are available for these diseases. Furthermore, most of these diseases are preceded by a functional phase (functional disorders or diseases), which are difficult to diagnose and treat. However, indicating the mucosal inflammation as a causal factor should yield a revolutionary change in treating any of the above listed diseases. This data explains the worldwide, government-funded research on the Human Microbiome Project. (NIH HMP Working Group, Peterson J, Garges S, Giovanni M, McInnes P, Wang L, Schloss J A, Bonazzi V, McEwen J E, Wetterstrand K A, Deal C, Baker C C, Di Francesco V, Howcroft T K, Karp R W, Lunsford R D, Wellington C R, Belachew T, Wright M, Giblin C, David H, Mills M, Salomon R, Mullins C, Akolkar B, Begg L, Davis C, Grandison L, Humble M, Khalsa J, Little A R, Peavy H, Pontzer C, Portnoy M, Sayre M H, Starke-Reed P, Zakhari S, Read J, Watson B, Guyer M. The NIH Human Microbiome Project, Genome Res. 2009 December; 19(12):2317-23. Epub 2009 Oct. 9). In summary: (i) in physiologic conditions, probiotics adhere to the mucosal surface and stimulating host immune system without inflammation, while (ii) in pathologic conditions—due to impaired mucosal structure and subsequent loss of probiotics—pathogenic bacteria passing through epithelial barrier affect the host's immune system and generate inflammation that is either local or systemic in nature.

Mostly on an empirical base, probiotics are widely used for treating mucosal inflammation, particularly in the gastrointestinal tract and in the vagina. Furthermore, some strains of probiotics were successfully tested for reducing blood cholesterol and glucose levels, known risk factors for cardiovascular diseases. However, in chronic and advanced form of these diseases even the continuous use of probiotics may be ineffective due to severe, treatment-resistant pathological alterations of the gastro-intestinal mucosa. In those cases, probiotics may actually aggravate local inflammatory diseases; furthermore, several cases of sepsis, due to probiotics, have also been reported, particularly in immune-compromised persons. From this brief description of probiotic use, it seems to be evident that the current clinical approach targets to restore the host-probiotics symbiosis through introducing billions of live probiotics, neglecting the contemporary alterations of the underlying mucosal epithelium.

Experimental and clinical studies showed that lysate of killed probiotics prevent lipid peroxidation and counteract inflammatory disease. This antioxidant effect was associated with anti-inflammatory and anti-atherogenic effects in humans. Putative molecular mechanism of action includes prevention of peroxidation of membrane phospholipids through introducing glycolysis-derived electrons into the plasma membrane redox system. Probiotics enhance anaerobic glycolysis by reducing levels of NADH. In addition, probiotics may also act through enhancing glutathione reductase activity thus reducing glutathione. (Mikelsaar M, Zilmer M. Lactobacillus fermentum ME-3—an antimicrobial and antioxidative probiotic. Microb Ecol Health Dis. 2009 April; 21(1):1-27. Epub 2009 Mar. 16). Importantly, cytoplasmic fraction of killed probiotics, but not the cell-wall fraction, was responsible for these effects suggesting a novel mechanism: likely gene-transfer from probiotics to host cells by phagocytosis resulting in enhanced anaerobic glycolysis of the host cells. This hypothesis is supported by observation that DNA from probiotic lysates bind to TLR 9. This TLR 9 signaling is essential in mediating the anti-inflammatory effect of probiotics. In an experimental model, live microorganisms were not required to attenuate colitis. (Rachmilewitz D, Karmeli F, Shteingart S, Lee J, Takabayashi K, Raz E. Immunostimulatory oligonucleotides inhibit colonic proinflammatory cytokine production in ulcerative colitis. Inflamm Bowel Dis. 2006 May; 12(5):339-45). Probiotics are facultative or obligate anaerobic microorganism and their DNA structure is highly conserved in mammals, humans included.

Nanotechnology has brought a variety of new possibilities into biological discovery and clinical practice. (Bhaskar S, Tian F, Stoeger T, Kreyling W, de la Fuente J M, Grazú V, Borm P, Estrada G, Ntziachristos V, Razansky D, Multifunctional Nanocarriers for diagnostics, drug delivery and targeted treatment across blood-brain barrier: perspectives on tracking and neuroimaging. Part Fibre Toxicol. 2010 Mar. 3; 7:3).

The strength of advanced drug delivery systems is their ability to alter the pharmacokinetics and biodistribution of the drug. Nanoparticles have unusual properties that can be taken advantage of to improve drug delivery. Where larger particles would have been cleared from the body before absorption, nanoparticles instead are taken up by the cells because of their size. Complex drug delivery mechanisms are being developed, including the ability to get drugs through cell membranes and into cell cytoplasm. Efficiency is important because many diseases depend upon processes within the cell and can only be impeded by drugs that make their way into the cell. In addition, nano-scaled carriers have revolutionized drug delivery, allowing for therapeutic agents to be selectively targeted on an organ, tissue and cell specific level, also minimizing exposure of healthy tissue to drugs.

Direct in vivo imaging of nanoparticles is another exciting recent field that can provide real-time tracking of nanocarriers. There is a range of systems suitable for in vivo imaging and monitoring of drug delivery, with an emphasis on most recently introduced molecular imaging modalities based on optical and hybrid contrast, such as fluorescent protein tomography and multispectral optoacoustic tomography

Nanomedical approaches to drug delivery center on developing nanoscale particles or molecules to improve drug bioavailability. Bioavailability refers to the presence of drug molecules where they are needed in the body and where they will do the most good. Drug delivery focuses on maximizing bioavailability both at specific places in the body and over a period of time.

Although both experimental and clinical studies supported enormous potentiality of nano-scaled particles as diagnostics, medicines, nutrients and cosmetics, accumulating evidence suggest severe adverse effects of this approach. One of the main problems is the cytotoxicity of nanoparticles used for diagnostic purposes or as carrier of active substances such as small molecules. Another adverse effect of nanopharmacology is the generation of ROS, which may cause cell damage and generate inflammation, thus both may severely compromise benefits of this approach. (See, e.g., De Jong W H, Borm P J. Drug delivery and nanoparticles: applications and hazards. Int J Nanomedicine. 2008; 3(2):133-49). (See also, Fröhlich E, Samberger C, Kueznik I, Absenger M, Roblegg E, Zimmer A, Pieber T R. Cytotoxicity of nanoparticles independent from oxidative stress. J Toxicol Sci. 2009 October; 34(4):363-75). (See also, Dusinska M, Dusinska M, Fjellsbø L. Magdolenova Z, Rinna A, Runden Pran E, Bartonova A, Heimstad E, Harju M, Tran L, Ross B, Juillerat L, Halamoda Kenzaui B, Marano F, Boland S, Guadaginini R, Saunders M, Cartwright L, Carreira S, Whelan M, Kelin Ch, Worth A, Palosaari T, Burello E, Housiadas C, Pilou M, Volkovova K, Tulinska J, Kazimirova A, Barancokova M, Sebekova K, Hurbankova M, Kovacikova Z, Knudsen L, Poulsen M, Mose T, Vilà M, Gombau L, Fernandez B, Castell J, Marcomini A, Pojana G, Bilanicova D, Vallotto D. Testing strategies for the safety of nanoparticles used in medical applications. Nanomedicine (Lond). 2009 August; 4(6):605-7).

### BRIEF SUMMARY OF EMBODIMENTS OF THE INVENTION

Various embodiments of the present invention provide methods for preparing nanoparticles from probiotics suitable for gene-repair cells in which the anaerobic metabolism is compromised.

In view of the above findings, it was postulated that the preparation and administration of nanoparticles from dead probiotics may guarantee better bioavailability and less adverse effects compared to live probiotics. In addition, the same nanotechnology used for preparing probiotics may be applied for preparing nanoparticles from other cells for potential medical, nutritional and cosmetic uses.

As set forth above, the current clinical approach targets to restore the host-probiotics symbiosis through introducing billions of live probiotics, neglecting the contemporary alterations of the underlying mucosal epithelium. However, embodiments of the invention suggest the opposite approach of first restoring the mucosal epithelium permitting spontaneous repopulation of mucosal surfaces by live probiotics. The use of nanoparticles derived from killed probiotics is a novel approach to rebuild the symbiosis of host and probiotics. The invention describes methods for in-vitro preparation of nanoparticles from probiotics mimicking as much as possible the in-vivo physiological processes.

Some embodiments of the present invention are directed toward a method for preparing probiotic nanoparticles from natural sources, comprising performing a biological preparation phase such as isolating any cells derived from either prokaryote or eukaryote cells, performing a chemical preparation phase such as performing an enzymatic procedure, or exposure to detergents, organic solvents, or antiseptic chemicals or heating or fractioned heating for killing or obtaining cell derived ingredients, performing a physical preparation phase such as performing ultrasonication, and performing a formulation preparation phase such as powderized drying.

In one embodiment, performing the biological preparation phase further comprises cultivating or fermenting the prokaryote or eukaryote cells. In addition, the step of performing the chemical preparation phase may entail performing an enzymatic procedure for killing or obtaining cell derived ingredients such as proteins, lipids, carbohydrates or nucleotides. The enzymatic procedure may include the use of proteases selected from the group consisting of: trypsin, chymotrypsin, pepsin, and papain. Additionally, the enzymatic procedure may include the use of lipases selected from the group consisting of: lingual lipase, gastric lipase, hepatic lipase, pancreatic lipase, bile-salt dependent lipase, and lysosomal lipase. In further embodiments, the enzymatic procedure may include the use of carbohydrases selected from the group consisting of: lysozyme, chymosin, amylases, glucanases, proteases, celluloses, pectinases, ligninases, lactases and xylanases. In other embodiments, the enzymatic procedure may include the use of nucleases selected from the group consisting of: deoxyribonuclease I and ribonuclease A.

Further chemical phase procedures include, but are not limited to: exposure to chemical substances (e.g., alcohol, formaldehyde, detergents, organic solvents, salt of heavy metals or any pharmacologically acceptable antiseptic substances), as well as heating or fractioned heating, specifically tyndallization or pasteurization. Tyndallization, or intermittent sterilization, essentially consists of boiling 3 to 5 times at 60-80° C. for 1 hour, separated by 24 hours to keep at 30-35° C. in an incubator. Pasteurization is a process of heating a food, usually liquid, to a specific temperature for a definite length of time, and then cooling it immediately. For example, milk is legally required to be heated to at least 72 degrees Celsius for at least 16 seconds and then cooled to 4 degrees Celsius.

In certain embodiments of the invention, performing a physical preparation phase may entail performing ultrasonication characterized by a frequency of 18 KHz to 1 MHz and a power of at least 100 watts. Alternatively, performing a physical preparation phase may comprise performing ultracentrifugation, disruption in bead, disruption using a colloid mill, disruption using French press, cryofracturing, osmotic shock, microwave exposure, gamma ray exposure, or UV-light exposure. In some embodiments, performing a formulation preparation phase may comprise powderized drying, desiccation to abolish hygroscopic nature of the dried lysate by the addition of glycogen or maltodextrin, or packaging and storage for preparing final products including powder, watery solutions, or lipid emulsions.

The method may further comprise using the prepared probiotic nanoparticles for medical, nutritional or cosmetic purposes. In addition, the method may comprise using the prepared probiotic nanoparticles for systemic or topical application. In some embodiments, the method may also entail applying the prepared probiotic nanoparticles enterally or parenterally. In further embodiments, the method may also comprise using the prepared probiotic nanoparticles for oral, intranasal, gastric or parenteral administrations. Additionally, the method may also entail using the prepared probiotic nanoparticles for the treatment of infective diseases, traumas, autoimmune disease, age-related diseases, malignancies, inherited disease, or connatal diseases, as well as functional disorders and diseases.

Other features and aspects of the invention will become apparent from the following detailed description, taken in conjunction with the accompanying drawings, which illustrate, by way of example, the features in accordance with embodiments of the invention. The summary is not intended to limit the scope of the invention, which is defined solely by the claims attached hereto.

### BRIEF DESCRIPTION OF THE DRAWINGS

The present invention, in accordance with one or more various embodiments, is described in detail with reference to the following figures. The drawings are provided for purposes of illustration only and merely depict typical or example embodiments of the invention. These drawings are provided to facilitate the reader's understanding of the invention and shall not be considered limiting of the breadth, scope, or applicability of the invention. It should be noted that for clarity and ease of illustration these drawings are not necessarily made to scale.

FIGS. 1A and 1B are first and second parts of a schematic illustrating an embodiment of a method for preparing nanoparticles using bifidobacterium as a substrate.

The figures are not intended to be exhaustive or to limit the invention to the precise form disclosed. It should be understood that the invention can be practiced with modification and alteration, and that the invention be limited only by the claims and the equivalents thereof.

### DETAILED DESCRIPTION OF THE EMBODIMENTS OF THE INVENTION

The present invention is generally directed toward methods for preparing nanoparticles from natural sources. More particularly, some embodiments relate to methods for preparing nanoparticles from probiotics, and to the use thereof either alone or in combination with pharmacologically compatible compounds for medical, nutritional and cosmetic purposes.

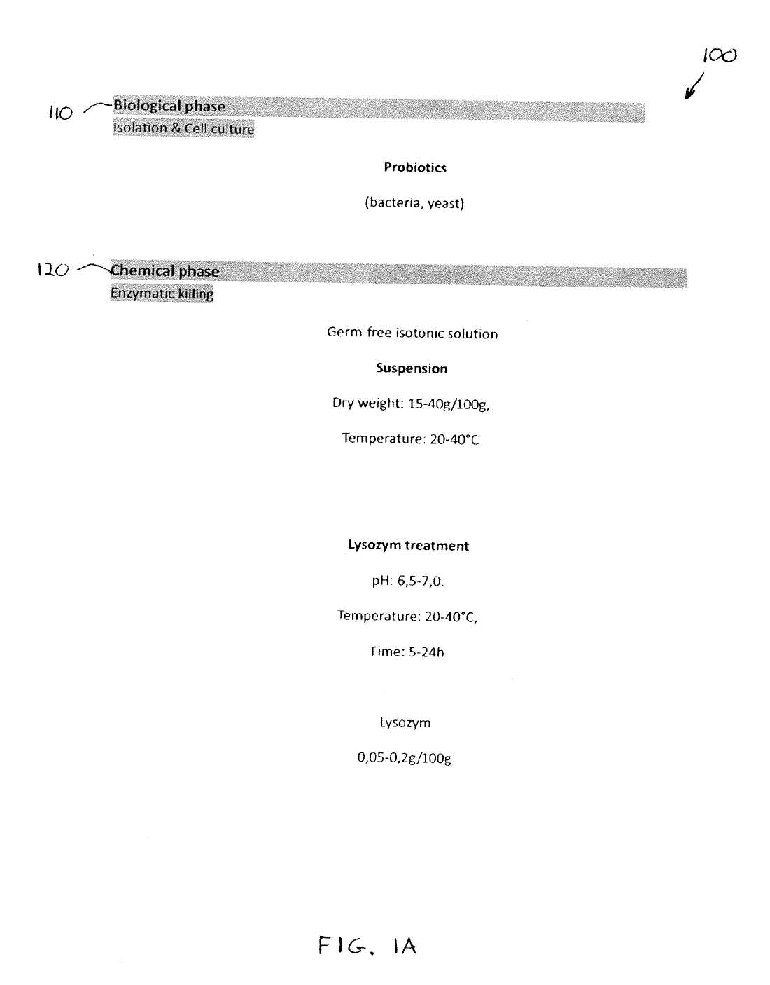

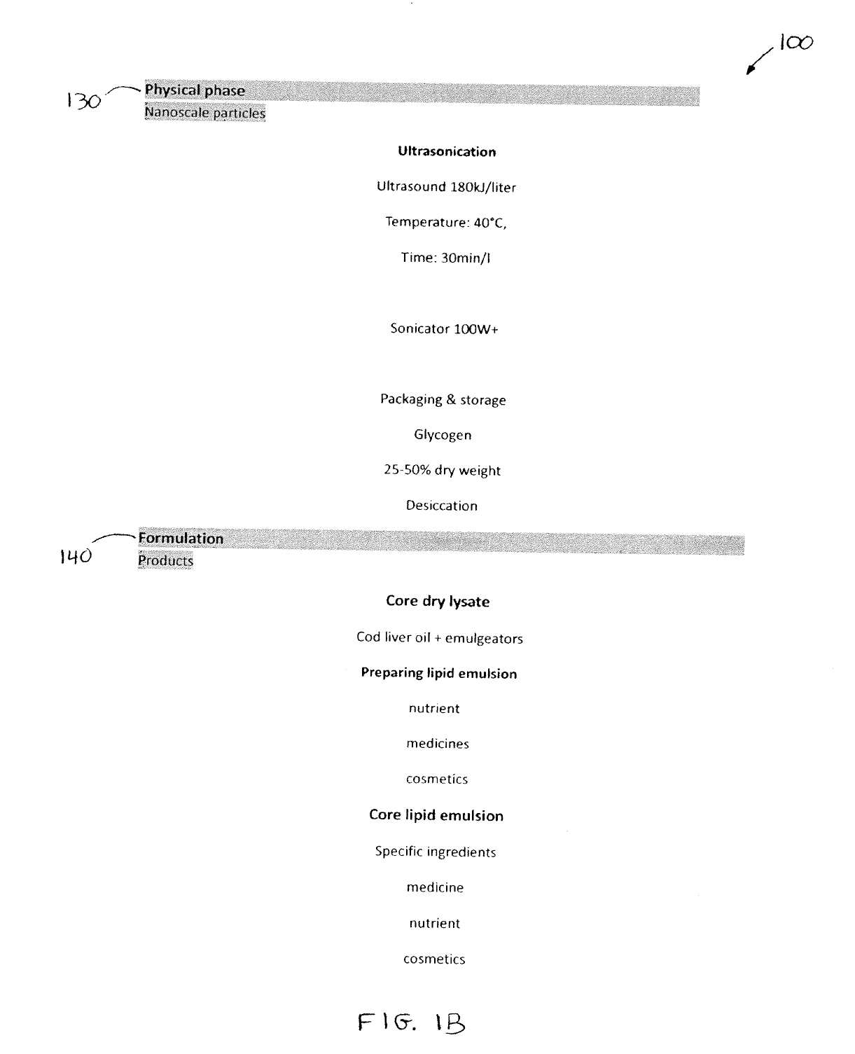

FIGS. 1A and 1B are first and second parts of a schematic illustrating an embodiment of a method 100 for preparing nanoparticles using bifidobacterium as a substrate. It should be noted, however, that any other probiotic bacteria or yeast may be employed without departing from the scope of the invention. In the illustrated embodiment, the method comprises performing a biological preparation phase 110 such as isolating any cells derived from either prokaryote or eukaryote cells, performing a chemical preparation phase 120 such as performing an enzymatic procedure for killing or obtaining cell derived ingredients, performing a physical preparation phase 130 such as performing ultrasonication, and performing a formulation preparation phase 140 such as powderized drying.

With further reference to FIGS. 1A and 1B, performing the biological preparation phase 110 may further comprise cultivating or fermenting the prokaryote or eukaryote cells. In addition, the step of performing the chemical preparation phase 120 may entail performing an enzymatic procedure for killing or obtaining cell derived ingredients such as proteins, lipids, carbohydrates or nucleotides. The enzymatic procedure may include the use of proteases selected from the group consisting of: trypsin, chymotrypsin, pepsin, and papain. Additionally, the enzymatic procedure may include the use of lipases selected from the group consisting of: lingual lipase, gastric lipase, hepatic lipase, pancreatic lipase, bile-salt dependent lipase, and lysosomal lipase. In further embodiments, the enzymatic procedure may include the use of carbohydrases selected from the group consisting of: lysozyme, chymosin, amylases, glucanases, proteases, celluloses, pectinases, ligninases, lactases and xylanases. In other embodiments, the enzymatic procedure may include the use of nucleases selected from the group consisting of: deoxyribonuclease I and ribonuclease A. In further embodiments, the chemical preparation phase may comprise exposure to heating or fractioned heating, specifically tyndallization or pasteurization as well as exposure to chemical substances (for example, alcohol, formaldehyde, detergents, organic solvents, salt of heavy metals or any pharmacologically acceptable antiseptic substances).

In certain embodiments of the invention, performing a physical preparation phase 130 may entail performing ultrasonication characterized by a frequency of 18 KHz to 1 MHz and a power of at least 100 watts. Alternatively, performing a physical preparation phase 130 may comprise performing ultracentrifugation, cryofracturing, osmotic shock, microwave exposure, gamma ray exposure, or UV-light exposure. In some embodiments, performing a formulation preparation phase 140 may comprise powderized drying, desiccation to abolish hygroscopic nature of the dried lysate by the addition of glycogen or maltodextrin, or packaging and storage for preparing final products including powder, watery solutions, or lipid emulsions

In some embodiments, the method 100 may further comprise using the prepared probiotic nanoparticles for medical, nutritional or cosmetic purposes. In addition, the method 100 may comprise using the prepared probiotic nanoparticles for systemic or topical application. In some embodiments, the method 100 may also entail applying the prepared probiotic nanoparticles enterally or parenterally. In further embodiments, the method 100 may also comprise using the prepared probiotic nanoparticles for oral, intranasal, gastric or parenteral administrations. Additionally, the method 100 may also entail using the prepared probiotic nanoparticles for the treatment of infective diseases, traumas, autoimmune disease, age-related diseases, malignancies, inherited disease, or connatal diseases.

Further details regarding the procedures used for preparing medicines, nutrients or cosmetics containing nanoparticles using method 100 including biological phase 110, chemical phase 120, physical phase 130 and formulation phase 140 will now be described.

Biological Phase

In some embodiments of the invention, the source of prime material for preparing nanoparticles may comprises bacteria, viruses, yeasts, any living animal or plant organism,and/or any part thereof. By way of example, the source may comprise cells from the root, trunk, leaves, flowers and/or fruits of plants, or products of these cells, as well as from any animal/human cells or products of these cells. One particular source of prime material may comprise stem cells, e.g., of donor origin or the user's own stem cells. Another particular source of prime material may comprise genetically engineered cells. Cells isolated from any of these sources may be directly used for preparing nanoparticles. In some embodiments, the cells may be multiplied in culture to reach industrial quantities. In further embodiments, the cells may be selected from the market of probiotics, or from the cell line bases.

Fermented products may also be used as prime material for preparing nanoparticles. Industrial fermentation involves the breakdown and re-assembly of biochemicals for industry, often in aerobic growth conditions. This is an intentional use of fermentation by microorganisms such as bacteria and fungi to make products useful to humans and animals. Various embodiments of the invention may entail the use of specific probiotic strains available in industrial quantities.

Chemical Phase

The preparation of nanoparticles may include enzymatic procedures, whereby a complex natural substance is exposed to any of the below enzymes, as sorted by their EC numbers. The EC number is the Enzyme Commission number, as determined by the International Union of Biochemistry and Molecular Biology. This numerical classification scheme for enzymes is based on the chemical reactions they catalyze. (11 Moss, 2006). (Moss, G. P. “Recommendations of the Nomenclature Committee,” International Union of Biochemistry and Molecular Biology on the Nomenclature and Classification of Enzymes by the Reactions they Catalyse. http://www.chem.gmul.ac.uk/iubmb/enzyme/. Retrieved 2006 Mar. 14.). The enzymes (along with their respective EC numbers) include: (i) Oxidoreductases (EC.1); Transferases (EC.2); (iii) Hydrolases (EC.3); (iv) Lyases (EC.4); (v) Isomerases (EC.5); (and (vi) Ligases (EC.6).

According to the various embodiments of the invention, preferable enzymes include: (i) digestion enzymes such as proteases and peptidases, which split proteins into amino acids; (ii) lipases, which split fat into three fatty acids and glycerol; (iii) carbohydrases, which split carbohydrates such as starch into sugars; and (iv) nucleases, which split nucleic acids into nucleotides. More preferable enzymes, include: (i) proteases such as trypsin, chymotrypsin, pepsin, and papain; (ii) lipases such as hepatic lipase, pancreatic lipase, bile-salt dependent lipase, lysosomal lipase, gastric lipase, lingual lipase, endothelial lipase, lipoprotein lipase, and phospholipases; (iii) carbohyrases such as lysozyme, amylases, glycoamylases, amyloglycosidases, betaglucanases, arabinoxylanases, glucanases, celluloses, pectinases, ligninases, chymosin, maltases, sucrases, lactases, and xylanases; and (iv) nucleases such as Deoxyribonuclease I, Ribonuclease A, HindIII nuclease, micrococcal nucleas, S1 nuclease, and P1 nuclease.

In further embodiments, the chemical preparation phase comprises fractioned heating, specifically tyndallization or pasteurization. Tyndallization, or intermittent sterilization, essentially consists of boiling 3 to 5 times at 60-80° C. for 1 hour, separated by 24 hours to keep at 30-35° C. in an incubator. Pasteurization is a process of heating a food, usually liquid, to a specific temperature for a definite length of e, and then cooling it immediately. For example, milk is legally required to be heated to at least 72 degrees Celsius for at least 16 seconds and then cooled to 4 degrees Celsius. The chemical preparation phase may also comprise exposure to detergents, organic solvents, or any pharmacologically acceptable antiseptic substances (e.g., alcohol, formaldehyde, heavy metals).

Physical Phase

The physical phase may entail fragmentation and desiccation. In particular, fragmentation may include, but is not limited to: (i) ultrasonication or ultrasonic cell disruption; (ii) cryo disruption; (iii) osmotic shock separation; and (iv) ultracentrifugation. With respect to ultrasonication or ultrasonic cell disruption, the treatment of microbial cells in suspension with inaudible ultrasound results in their inactivation and disruption. Ultrasonication utilizes the rapid sinusoidal movement of a probe within the liquid. It is characterized by high frequency (18 kHz-1 MHz), small displacements (less than about 50 μm), moderate velocities (a few m s-1), steep transverse velocity gradients (up to 4,000 s-1) and very high acceleration (up to about 80,000 g). In some embodiments, desiccation may entail drying and pulverization including air-drying and freeze-drying (lyophilisation). In further embodiments, desiccation may comprise mixing with desiccants. Dried lysate from probiotics and other cellular sources may be hygroscopic. This characteristic may create difficulties in the storage, such that vacuum storage or freezing may be needed. However, the addition of an adequate quantity of desiccant substances may offer a solution. Such desiccants may include polysaccharides such as starch, maltodextrin, and cellulose. The amount of added desiccant may vary from 1% to 200% of dry-weight of lysate. The aim of this procedure to maintain biological characteristics of the lysate for further procedures.

Formulation Phase

Dried lysate from probiotics and other cellular sources mixed with desiccant may be further processed for at least in three forms for medical, nutritional or cosmetic uses with appropriate excipients, including dry powder (e.g., tablets, capsules), water solution (e.g., injections, eye drops, lotion, spray, gel), and lipid emulsion (e.g., soft gel, injection, cream, spray), as well as for nutritional uses either in aqueous solution (e.g., in fruit juices, and other beverages) and in culinary products (e.g., in olive oil, butter, etc.). In further embodiments, the formulation phase includes the isolation or separation of desired fragments of cells destined for use in the further phases of preparation, e.g. for medical, nutritional or cosmetic use. These isolation or separation methods may be selected from conventional laboratory and industrial technologies.

Co-owned U.S. patent application Ser. No. 12/675,504, entitled Compositions and Methods for Inhibiting Inflammation, is directed toward: (i) compositions of killed probiotics and omega 3 fatty acids (eventually with combination of pharmacologically acceptable substances); (ii) methods for formulating lipid emulsion in which active ingredients form nanoparticles; and (iii) use of these compositions for preventing, attenuating or treating inflammatory diseases either of low-grade or manifest in nature with topical or systemic administration. This patent application is hereby incorporated herein in its entirety. The patent application discloses that nano-sized particles of killed (dead) probiotics are used instead of live probiotics. The nano-sized particles of killed probiotics enter into the target host cells by phagocytosis. In this way, DNA/RNA from selected probiotics may be transferred into host cells where they contribute to gene repair. Probiotics' genes improve host anaerobic metabolism (glycolysis), resulting in enhanced generation of NADH and NADPH and releasing electrons into the cell membranes, specifically into the Plasma Membrane Redox System (PMRS). This mechanism prevents the release of lipid peroxides from plasma membrane phospholipids, the earliest step of inflammation (i.e., elementary inflammation), inhibiting the generation of prostaglandin and leukotriene, which are known mediators of manifest inflammation.

The '504 application further discloses that the addition of omega 3 fatty acids enhances probiotics' effects in synergy. The nano-sized particles derived from probiotics are dispersed in omega 3 fatty acids forming either “water in oil” or “oil in water” emulsion suitable for both topical and systemic uses in a wide range of inflammatory diseases. In addition, the application teaches that the mixture of probiotics and omega 3 represents a novel approach for treating a wide range of diseases in which inflammation plays a pathogenic role, such as age-related diseases, metabolic diseases, autoimmune diseases, traumas and cancer.

Research according to the present invention has revealed that the anti-inflammatory effect of probiotics is coupled to the renewal processes of postmitotic cells. In particular, extensive studies were conducted on age-related macular degeneration, which is a common neurodegenerative eye-disease affecting the central area of the retina. This disease, in addition to its own importance, is an extremely suitable model to learn more on the renewal (turnover) mechanism of other postmitotic cells. Postmitotic cells, like neuronal cells, muscle cells, bone-cells, lost their capacity to multiply by cell division (mitosis). These cells perform continuous renewal at molecular levels. This cellular renewal mechanism comprises three types of processes: (i) autophagy, which is the uptake and enzymatic digestion of worn-out materials; (ii) recycling of most of suitable molecules from the autophagy; and (iii) burning of substances not used for recycle. In normal conditions, burned substances are replaced from the diet to maintain the balance.

Further research according to the invention has revealed that the three cellular renewal processes are coupled to the anaerobic cellular metabolism, also known as anaerobic glycolysis. Impairment of the anaerobic metabolism may compromise each of these processes, resulting in incomplete renewal of the involved cells and accumulation of metabolic byproduct either inside these cells or nearby the cells. Among the metabolic byproducts, reactive oxygen spices (ROS) are the best characterized. Current concepts on age-related diseases assign a central role to the excessive generation of ROS responsible for generation of inflammation and cell death, as well as for the subsequent diseases. According to the invention, it has been concluded from these observations that the administration of probiotic nanoparticles enhances anaerobic metabolism that inhibit inflammation and at the same time improves cell renewal. Clinically these two effects play a crucial role in preserving health or improving healing in diseases.

U.S. patent application Ser. No. 12/675,504 is directed toward the size of ingredient particles, i.e. nanoparticles for both topical and systemic uses. The present application includes similarly sized nanoparticles, but does not require lipid emulsion such that nanoparticles may be used alone. Additionally, according to the present invention, essential fatty acids, such as omega 3 fatty acids, are merely optional components of the composition, together with essential amino acids, vitamins, trace elements and several other pharmacologically acceptable substances. In the '504 application, the origin of DNA/RNA is restricted to selected probiotics. In the present invention, DNA/RNA may come from any living prokaryote and eukaryote cells, or from ex-vivo synthesis to improve any gene-dysfunction. The prokaryotes are a group of organisms that lack a cell nucleus, or any other membrane-bound organelles. They differ from the eukaryotes, which have a cell nucleus. The prokaryotes are divided into two domains: the bacteria and the archaea. Archaea were recognized as a domain of life in 1990. These organisms were originally thought to live only in inhospitable conditions such as extremes of temperature, pH, and radiation, but have since been found in all types of habitats.

In the '504 application, the selection criteria for probiotics include their contribution to the anaerobic glycolysis. In other words, the probiotics employed have genes related to the anaerobic glycolysis to improve host anaerobic glycolysis. In the present invention, the selection criteria are variable depending on the target and scope of intervention. This process may be referred to herein as “horizontal gene repair” (HGR), indicating the scope of this intervention. From ethical viewpoint, this is “gene-repair” is distinguished from “gene-substitution.” The procedure intends to improve (repair) gene functions/expressions without changing the individual's genome.

According to the various embodiments of the invention, the use of probiotics is not restricted to inflammatory and/or inflammation related diseases, but includes virtually all diseases caused or aggravated by gene-dysfunction. Some embodiments involve the use of probiotic nanoparticles as an adjuvant in nanomedicine. This novel application is suitable to abolish or reduce toxicity of other nanoparticles introduced for therapeutic or diagnostic purposes, or used as nano-carrier for small molecules. Additional embodiments of the invention permit novel applications for nanoparticles in all diseases in which cell renewal and regeneration of cell compartments is impaired due to inadequate metabolic support, such diseases include, but are not limited to: (i) age-related diseases; (ii) traumas (improved regeneration of damaged cells); (iii) infectious diseases (improved regeneration); (iv) immune-autoimmune disease; and (v) cancer and other malignancies. Further embodiments of the invention permit novel applications for nanoparticles in all functional diseases in which the energy consuming cellular or molecular mechanisms are impaired due to inadequate metabolic support. Such diseases include, but are not limited to: (i) functional brain diseases (anxiety, depression, panic disease, reduced stress resistance, chronic fatigue syndrome, fibromyalgia, and other poorly characterized types of mood and behavior diseases and personality disorders), and (ii) chanelopathies (dysfunction of ion-channels of neuronal, muscular or any other cells). However, it should be noted that these functional disorders or diseases are almost always the earliest phase of organic diseases listed above (e.g., age-related diseases, autoimmune diseases, cancer). Accordingly, the present invention extends the probiotics' mediated support to all cellular processes which need energy. Consequently, the therapeutic use of probiotics' nanoparticles, in addition to inflammatory diseases, is also extended to all diseases in which impaired cellular metabolism play a role.

One embodiment of the invention involves the elaboration of an industrial technology for preparing nanoparticles from living cells destined to use for medical, nutritional and cosmetic purposes. The combination of enzymatic digestion with the habitual physical fragmentation increases the efficacy of this technology as the action of lysozyme or other enzymes mimics natural killing and elaboration of probiotics.

Another embodiment of the invention entails the preparation of nanoparticles from any prokaryote and eukaryote cells.

A further embodiment of the invention involves the co-administration of nano-probiotics with other nanoparticles for preventing cytotoxicity, particularly in chemotherapy for cancer and other malignancies.

While various embodiments of the present invention have been described above, it should be understood that they have been presented by way of example only, and not of limitation. Likewise, the various diagrams may depict an example architectural or other configuration for the invention, which is done to aid in understanding the features and functionality that can be included in the invention. The invention is not restricted to the illustrated example architectures or configurations, but the desired features can be implemented using a variety of alternative architectures and configurations. Indeed, it be apparent to one of skill in the art how alternative functional, logical or physical partitioning and configurations can be implemented to implement the desired features of the present invention. In addition, a multitude of different constituent module names other than those depicted herein can be applied to the various partitions. Additionally, with regard to flow diagrams, operational descriptions and method claims, the order in which the steps are presented herein shall not mandate that various embodiments be implemented to perform the recited functionality in the same order unless the context dictates otherwise.

Although the invention is described above in terms of various exemplary embodiments and implementations, it should be understood that the various features, aspects and functionality described in one or more of the individual embodiments are not limited in their applicability to the particular embodiment with which they are described, but instead can be applied, alone or various combinations, to one or more of the other embodiments of the invention, whether or not such embodiments are described and whether or not such features are presented as being a part of a described embodiment. Thus, the breadth and scope of the present invention should not be limited by any of the above-described exemplary embodiments.

Terms and phrases used in this document, and variations thereof, unless otherwise expressly stated, should be construed as open ended as opposed to limiting. As examples of the foregoing: the term “including” should be read as meaning “including, without limitation” or the like; the term “example” is used to provide exemplary instances of the item in discussion, not an exhaustive or limiting list thereof; the terms “a” or “an” should be read as meaning “at least one,” “one or more” or the like; and adjectives such as “conventional,” “traditional,” “normal,” “standard,” “known” and terms of similar meaning should not be construed as limiting the item described to a given time period or to an item available as of a given time,but instead should be read to encompass conventional, traditional, normal, or standard technologies that may be available or known now or at any time in the future. Likewise, where this document refers to technologies that would be apparent or known to one of ordinary skill in the art, such technologies encompass those apparent or known to the skilled artisan now or at any time in the future.

The presence of broadening words and phrases such as “one or more,” “at least,” “but not limited to” or other like phrases in some instances shall not be read to mean that the narrower case is intended or required in instances where such broadening phrases may be absent. The use of the term “module” does not imply that the components or functionality described or claimed as part of the module are all configured in a common package. Indeed, any or all of the various components of a module, whether control logic or other components, can be combined in a single package or separately maintained and can further be distributed in multiple groupings or packages or across multiple locations.

Additionally, the various embodiments set forth herein are described in terms of exemplary block diagrams, flow charts and other illustrations. As will become apparent to one of ordinary skill in the art after reading this document, the illustrated embodiments and their various alternatives can be implemented without confinement to the illustrated examples. For example, block diagrams and their accompanying description should not be construed as mandating a particular architecture or configuration.

What is claimed is:

1. A method for preparing probiotic nanoparticles from natural sources, comprising:
performing a biological preparation phase on a plurality of cells;
performing a chemical preparation phase on the cells to obtain cell derived ingredients;
performing a physical preparation phase on the derived cell ingredients to produce dried lysate; and
performing a formulation preparation phase on the dried lysate.

2. The method of claim 1 wherein performing the biological preparation phase comprises isolating any cells derived from either prokaryote or eukaryote cells.

3. The method of claim 2, wherein performing the biological preparation phase further comprises cultivating or fermenting the prokaryote or eukaryote cells.

4. The method of claim 1, wherein performing the chemical preparation phase comprises performing an enzymatic, heating or chemical procedure for killing and/or obtaining cell derived ingredients.

5. The method of claim 4, wherein the cell derived ingredients comprise proteins, lipids, carbohydrates, nucleotides, or a combination thereof

6. The method of claim 4, wherein the enzymatic procedure includes the use of proteases selected from the group consisting of: trypsin, chymotrypsin, pepsin, and papain.

7. The method of claim 4, wherein the enzymatic procedure includes the use of lipases selected from the group consisting of: lingual lipase, gastric lipase, hepatic lipase, pancreatic lipase, bile-salt dependent lipase, and lysosomal lipase.

7. The method of claim 4, wherein the enzymatic procedure includes the use of carbohydrases selected from the group consisting of: lysozyme, chymosin, amylases, glucanases, proteases, celluloses, pectinases, ligninases, lactases and xylanases.

8. The method of claim 4, wherein the enzymatic procedure includes the use of nucleases selected from the group consisting of: deoxyribonuclease I and ribonuclease A.

9. The method of claim 1, wherein performing the chemical preparation phase comprises heating, fractioned heating, or exposure to chemicals.

10. The method of claim 1, wherein performing a physical preparation phase comprises performing ultrasonication characterized by a frequency of 18 KHz to 1 MHz and a power of at least 100 watts.

11. The method of claim wherein performing a physical preparation phase comprises performing ultracentrifugation, disruption in bead mill, disruption using a colloid mill, disruption using French press, cryofracturing, osmotic shock, microwave exposure, gamma ray exposure, or UV-light exposure or.

15. The method of claim 1, wherein performing a formulation preparation phase comprises powderized drying, desiccation to abolish hygroscopic nature of the dried lysate by the addition of glycogen or maltodextrin, or packaging and storage for preparing final products including powder, watery solutions, or lipid emulsions

16. The method of claim 1, further comprising using the prepared probiotic nanoparticles for medical, nutritional or cosmetic purposes.

17. The method of claim 1, further co sing using the prepared probiotic nanoparticles for systemic or topical application.

18. The method of claim 1, further comprising applying the prepared probiotic nanoparticles enterally or parenterally.

19. The method of claim 1, further comprising using the prepared probiotic nanoparticles for oral, intranasal, gastric or parenteral administrations.

20. The method of claim 1, further comprising using the prepared probiotic nanoparticles for the treatment of infective diseases, traumas, autoimmune disease, age-related diseases, malignancies, inherited diseases, or connatal diseases, as well as functional diseases and disorders of the nervous system, endocrine/hormonal system, immune system, and skeleto-muscular system.
